(19) United States
(12) Patent Application Publication
(10) Pub. No.: US 2020/0113816 A1
(43) Pub. Date: Apr. 16, 2020

(54) REACTIONS TRIGGERED BY COOLING-INDUCED GEL-TO-SOL TRANSITION OF THERMORESPONSIVE POLYMERS

(71) Applicant: Vanderbilt University, Nashville, TN (US)

(72) Inventor: Leon M. Bellan, Nashville, TN (US)

(21) Appl. No.: 16/653,883

(22) Filed: Oct. 15, 2019

Related U.S. Application Data
(60) Provisional application No. 62/745,795, filed on Oct. 15, 2018.

(51) Int. Cl. A61K 9/00; A61K 9/06; A61K 47/32; A61K 31/445; A61K 47/34; A61K 47/36

(52) U.S. Cl. CPC A61K 9/0004; A61K 9/06; A61K 47/32; A61K 47/36; A61K 47/34; A61K 9/0019; A61K 31/445

(57) ABSTRACT

Provided are pharmaceutical compositions, which include a gel comprising a thermoresponsive polymer, the polymer being soluble in water at a temperature of 37° C. or lower; and a therapeutic compound, or a pharmaceutically acceptable salt thereof, dispersed in the gel. Also provided are methods of delivering a therapeutic compound, which include administering the disclosed pharmaceutical composition to a subject in need thereof (e.g., by subcutaneous administration).

### CROSS-REFERENCE TO RELATED APPLICATIONS

This application claims the benefit of and priority to U.S. Provisional Application No. 62/745,795, filed on Oct. 15, 2018, the entire contents of which are hereby incorporated by reference.

### STATEMENT OF GOVERNMENT INTEREST

This invention was made with government support under Grant number 1506717 awarded by the National Science Foundation (NSF). The government has certain rights in the invention.

### INTRODUCTION

There is a critical need for on-demand, patient triggered pain relief to reduce or avoid the need for systemic therapy using opioids, which has significant risk for addiction and overdose. The growing severity of opioid addiction and abuse throughout the United States has led to increased interest in more targeted and controlled approaches to pain management. In particular, prolonged duration local anesthesia has the potential to treat both chronic and acute pain without the many disadvantages and risks of systemic therapies. It would be highly desirable for patients themselves to controllably and non-invasively trigger the release of an anesthetic to manage their own pain. To enable such capability, several approaches have been developed; the stimuli used in these systems, however, must be provided by complex apparatus that can send the necessary energy in photothermal, ultrasound, or electromagnetic form to trigger release.

Thus, there remain a need to develop alternative stimulus-response delivery methods.

### SUMMARY

In one aspect, the present disclosure provides a pharmaceutical composition comprising: a gel comprising a thermoresponsive polymer, the polymer being soluble in water at a temperature of 37° C. or lower; and a therapeutic compound, or a pharmaceutically acceptable salt thereof, dispersed in the gel.

In another aspect, the present disclosure provides a method of delivering a therapeutic compound, the method comprising: administering the pharmaceutical composition described herein to a subject in need thereof. The compositions may be administered, for example, subcutaneously, intradermally, or intramuscularly.

In another aspect, the present disclosure provides a method of inducing a chemical reaction, the method comprising reducing a temperature of a reaction system comprising: a first gel comprising a thermoresponsive polymer and a first reactant in the first gel; and a second gel comprising a thermoresponsive polymer and a second reactant in the second gel; wherein each polymer is soluble in water below a lower critical solution temperature (LCST); thereby causing each gel to transition to a sol state and mix, thereby allowing the first reactant and the second reactant to mix, and thereby initiating the chemical reaction.

### BRIEF DESCRIPTION OF THE DRAWINGS

FIG. 1. shows a schematic illustration of cooling triggered release of therapeutic molecules bound in a physically crosslinked thermoresponsive gel near the skin surface.

FIGS. 2A and 2B show typical thermoresponsive behavior of commercially available PNIPAM of molecular weight of 2 kDa (FIG. 2A) and 30 kDa (FIG. 2B). The polymers reversibly gel above the LCST (32° C.) and dissolve when cooled below this temperature.

FIG. 2C shows GFP-expressing Human Umbilical Vein Endothelial Cells (HUVECs) cultured in the presence of a PNIPAM gel (Mw 30 kDa) loaded with Nile Red (5% gel concentration, 800 ng/ml Nile Red concentration), which are incubated at 37° C. (top) and subsequently cooled to room temperature (bottom), at which point the dye is released and taken up by the cells, causing a dramatic increase in red fluorescence.

FIG. 3 shows the representative results of an in vitro experiment of cooling-triggered delivery of Nile Red. T1—immediately after adding warm gel to cell culture, T2—after 24 hours of culture in 37° C., T3—after removing from incubator and equilibrating at room temperature for 30 min, and T4—after removing from incubator and equilibrating at room temperature for 60 min. Scale bar is 400. Experiments of gels without dye (“No dye”) and experiments without gel or dye (“No gel or dye”) are used as controls. Imaging parameters (e.g., gain, exposure time) and brightness/contrast settings are kept constant.

### DETAILED DESCRIPTION

Disclosed herein are pharmaceutical compositions have a thermoresponsive polymer and a therapeutic compound, such as an anesthetic compound, which may be released upon cooling. The pharmaceutical compositions may be included, for example, in an implant device, and may be subcutaneously administered to deliver the therapeutic compound to a patient. In particular embodiments, the present disclosure relates to a composition that may enable cooling-triggered release of anesthetics from an injectable thermogelling formulation. The cooling may include application of ice or a cold pack to the skin, which may trigger prolonged pain relief. Advantageously, this present compositions and method may use a stimulus that patients already associate with temporary pain relief (cooling), and avoid the need for complex apparatus to deliver energy in the appropriate form for triggering release of medication.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkyl” as used herein, means a straight or branched chain saturated hydrocarbon. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “thermoresponsive polymer” refers to a polymer having a physical or chemical property that undergoes a discontinuous or binary change in a temperature-dependent manner. For example, the physical conformation or polarity of a thermoresponsive polymer may change in a temperature-dependent manner, and the thermoresponsive polymer exhibits a first conformation below a threshold temperature and a second, substantially different conformation above the threshold temperature. In a particular example, a thermoresponsive polymer exhibits an expanded coil or chain confirmation below a lower critical solution temperature (LCST) temperature, and exhibits a compact or globular conformation above such threshold temperature. The thermoresponsive behavior also may be observed when such polymer is placed in an aqueous environment. For example, the polymer may be in contact with, or is embedded in a hydrated matrix (such as an aqueous solution or a hydrogel) and may exhibit a thermoresponsive behavior such that they are hydrophobic and insoluble above the LCST, and become hydrophilic and soluble below the LCST. When warmed, the thermoresponsive polymer may undergo a reversible hydrated-coil to hydrophobic-globule transition as the temperature increases beyond the LCST, and above a critical concentration this results in a macroscopic, reversible sol→4 gel transition.

The thermoresponsive polymers as described herein may form a physically crosslinked gel when heated above the LC ST at a high enough concentration (sol→4 gel transition); this gel may disassemble and the polymers may dissolve when cooled below the LCST (gel→4 sol transition). The term “physically crosslinked” refers to the formation of a gel by a thermoresponsive polymer as described herein without the formation of any covalent bonds between individual polymer molecules. The physically crosslinked hydrogel is understood herein to be distinguishable from a chemically crosslinked hydrogel formed by a thermoresponsive polymer, which de-swells and ejects the absorbed molecules along with water when heated to a temperature above the LCST.

The term “administering” and/or “administer” as used herein refer to any route for delivering a pharmaceutical composition to a subject. Routes of delivery may include non-invasive peroral (through the mouth), topical (skin), transmucosal (nasal, buccal/sublingual, vaginal, ocular and rectal) and inhalation routes, as well as parenteral routes, and other methods know in the art. Parenteral refers to a route of administration that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. For administration via the parenteral route, the compositions may be in the form for infusion or for injection under the skin of the subject.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated

### 2. PHARMACEUTICAL COMPOSITION

In one aspect, provided is a pharmaceutical composition comprising:

a gel comprising a thermoresponsive polymer, the polymer being soluble in water at a temperature of 37° C. or lower; and

a therapeutic compound, or a pharmaceutically acceptable salt thereof, dispersed in the gel.

In some embodiments, the gel is a hydrogel. For example, the hydrogel may be formed by mixing the thermoresponsive polymer and the therapeutic compound in an aqueous solution at a temperature below the LCST of the polymer, followed by incubating the mixture at a temperature above the LCST. During the incubation, the polymer molecules are physically-crosslinked to form a macroscopic hydrogel, with the molecules of the therapeutic compound dispersed therein.

The thermoresponsive polymer disclosed herein may undergo a gel→sol transition upon cooling and a reverse sol→gel transition upon heating in vivo. The gel→sol and sol→gel transitions may be at least partially reversible in response to the cooling and heating. The transitions may be repeatable in response to multiple cycles of heating and cooling.

The thermoresponsive polymer may comprise a monomer selected from the group consisting of N-alkyl acrylamide, N,N-dialkyl acrylamide, N-acryloylpyrrolidine, N-acryloylpiperidine, N-vinyl caprolactam, N-vinyl propylacetamide, N-vinyl-5-methyl-2-oxazolidone, N-vinyl isobutyramide, N-acryloyl-L-proline methyl ester, N-acryloyl-4-trans-hydroxy-L-proline methyl ester, methyl 2-propionamidoacrylate, methyl 2-isobutyracrylate, acrylic acid, glucose, O-methyl glucose, lactic acid, glycolic acid, vinyl acetate, ethylene glycol, propyleneoxide, vinylether, alkylglycidylethers, phosphoester, and a combination thereof.

The acrylamide monomer may be optionally substituted with an alkyl group, such as methylacrylamide. Examples of N-alkyl acrylamide include, but are not limited to N-ethylacrylamide, N-ethylmethacrylamide, N-n-propylacrylamide, N-n-propylmethacrylamide, N-isopropylacrylamide, N-isopropylmethacrylamide. Examples of N,N-dialkyl acrylamide include, but are not limited to N,N′-ethylmethylacrylamide and N,N′-diethylacrylamide. Examples of vinylether include, but are not limited to, methylvinylether, methylvinylether, 2-ethoxyethylvinylether, 2-(2-ethoxy)ethoxyethylvinylether, and 4-hydroxybutylvinylether. Examples of alkylglycidylethers include, but are not limited to methyl glycidyl ether, ethyl glycidyl ether, and ethoxyethyl glycidyl ether. Examples of phosphoesters include, but are not limited to, 2-ethoxy-2-oxo-1,3,2-dioxaphospholane and 2-isopropoxy-2-oxo-1,3,2-dioxaphospholane.

In some embodiments, the thermoresponsive polymer comprises a monomer selected from the group consisting of N-ethylacrylamide, N-ethylmethacrylamide, N,N′-ethylmethylacrylamide, N,N′-diethylacrylamide, N-n-propylacrylamide, N-n-propylmethacrylamide, N-isopropylacrylamide, N-isopropylmethacrylamide, and a combination thereof.

In some embodiments, the thermoresponsive polymer is a homopolymer. In some embodiments, the thermoresponsive polymer is a copolymer.

In some embodiments, the thermoresponsive polymer comprises poly(N-isopropylacrylamide) (PNIPAM) having an average molecular weight (Mw) of about 1 kDa to about 100 kDa. Suitable poly(N-isopropylacrylamide) may have an average molecular weight of greater than 10 kDa, greater than 20 kDa, greater than 30 kDa, greater than 40 kDa, greater than 50 kDa, greater than 60 kDa, greater than 70 kDa, greater than 80 kDa, or greater than 90 kDa. Suitable poly(N-isopropylacrylamide) may have an average molecular weight of less than 100 kDa, less than 90 kDa, less than 80 kDa, less than 70 kDa, less than 60 kDa, less than 50 kDa, less than 40 kDa, less than 30 kDa, less than 20 kDa, less than 10 kDa, or less than 5 kDa. In some embodiments, the poly(N-isopropylacrylamide) has an average molecular weight of about 5 kDa to about 85 kDa, including for example, about 10 kDa to about 80 kDa, about 20 kDa to about 80 kDa, about 30 kDa to about 80 kDa, about 10 kDa to about 60 kDa, about 20 kDa to about 60 kDa, about 30 kDa to about 60 kDa, or about 40 to about 60 kDa. In some embodiments, the poly(N-isopropylacrylamide) has an average molecular weight of about 10 kDa to about 60 kDa, or about 30 kDa to about 60 kDa. In some embodiments, the poly(N-isopropylacrylamide) has an average molecular weight of about 10 kDa, about 20 kDa, about 40 kDa, about 60 kDa, about 70 kDa, about 80, or about 90 kDa. In particular embodiments, the poly(N-isopropylacrylamide) has an average molecular weight of about 10 kDa, about 40 kDa, or about 85 kDa.

Other suitable acrylamide-based thermoresponsive polymer include, for example, poly(N,N-diethylacrylamide), poly[2-(dimethylamino)ethyl methacrylate], or a copolymer of N-isopropylacrylamide with one or more monomer selected from the group consisting of acrylamide, N-tert-butylacrylamide, acrylic acid, ethylene glycol, and allylamine.

In some embodiments, the thermoresponsive polymer comprises a monomer selected from the group consisting of vinyl caprolactam, vinyl acetate, ethylene glycol, and a combination thereof. In some embodiments, the thermoresponsive polymer is a poly(N-vinylcaprolacatam). In some embodiments, the thermoresponsive polymer is a copolymer comprising vinyl caprolactam, vinyl acetate, ethylene glycol, or a combination thereof. In some embodiments, the thermoresponsive polymer is a copolymer comprising vinyl caprolactam and vinyl acetate, or a copolymer comprising vinyl caprolactam and ethylene glycol. In some embodiments, the thermoresponsive polymer is a copolymer comprising vinyl caprolactam, vinyl acetate, and ethylene glycol. In some embodiments, the thermoresponsive polymer is a graft copolymer comprising vinyl caprolactam, vinyl acetate, ethylene glycol, or a combination thereof. In some embodiments, the thermoresponsive polymer is a graft copolymer comprising vinyl caprolactam and vinyl acetate, or a graft copolymer comprising vinyl caprolactam and ethylene glycol. In particular embodiments, the thermoresponsive polymer is a graft copolymer comprising vinyl caprolactam, vinyl acetate, and ethylene glycol. For example, the thermoresponsive polymer may be a polyvinyl caprolactam-polyvinyl acetate-polyethylene glycol graft copolymer, such as the commercial product Soluplus®.

In some embodiments, the thermoresponsive polymer comprises a monomer selected from the group consisting of glucose, O-methyl glucose, lactic acid, glycolic acid, caprolactone, and a combination thereof. For example, the thermoresponsive polymer may be cellulose, methylcellulose, poly(N-vinylcaprolacatam), polylactic acid, polyglycolic acid, poly(lactic-co-glycolic acid), or polycaprolactone.

In some embodiments, the thermoresponsive polymer comprises poly(N-isopropylacrylamide), poly(N,N-diethylacrylamide), poly[2-(dimethylamino)ethyl methacrylate], cellulose, methylcellulose, poly(N-vinylcaprolacatam), polylactic acid, polyglycolic acid, poly(lactic-co-glycolic acid), polycaprolactone, a copolymer of N-isopropylacrylamide with one or more monomer selected from the group consisting of acrylamide, N-tert-butylacrylamide, acrylic acid, and allylamine, a copolymer comprising at least one monomer selected from group consisting of vinyl caprolactam, vinyl acetate, and ethylene glycol, or a combination thereof. Suitable thermoresponsive polymer may include those described in Aseyev et al. (Non-ionic Thermoresponsive Polymers in Water, Adv. Polym. Sci., 2011, 242: 29-89), which is incorporated herein by reference in its entirety.

In some embodiments, the thermoresponsive polymer is soluble in water at a temperature of 37° C. or lower, 36° C. or lower, 35° C. or lower, 34° C. or lower, 33° C. or lower, 32° C. or lower, 31° C. or lower, 30° C. or lower, 29° C. or lower, 28° C. or lower, 27° C. or lower, 26° C. or lower, 25° C. or lower, 24° C. or lower, 23° C. or lower, 22° C. or lower, 21° C. or lower, 20° C. or lower, 19° C. or lower, or 18° C. or lower. In some embodiments, the thermoresponsive polymer is soluble in water at a temperature of 32° C. or lower, 28° C. or lower, or 24° C. or lower. In particular embodiments, the thermoresponsive polymer is soluble in water at a temperature of 32° C. or lower.

In some embodiments, the thermoresponsive polymer is insoluble in water at a temperature above 19° C., above 20° C., above 21° C., above 22° C., above 23° C., above 24° C., above 25° C., above 26° C., above 27° C., above 28° C., above 29° C., above 30° C., above 31° C., above 32° C., above 33° C., above 34° C., above 35° C., or above 36° C. In some embodiments, the thermoresponsive polymer is insoluble in water at a temperature above 28° C., above 30° C., above 32° C., or above 34° C. In particular embodiments, the thermoresponsive polymer is insoluble in water at a temperature above 32° C.

In some embodiments, the thermoresponsive polymer is insoluble in water at a temperature above 37° C., and is soluble in water at a temperature of 37° C. or lower. In some embodiments, the thermoresponsive polymer is insoluble in water at a temperature above 36° C., and is soluble in water at a temperature of 36° C. or lower. In some embodiments, the thermoresponsive polymer is insoluble in water at a temperature above 34° C., and is soluble in water at a temperature of 34° C. or lower. In some embodiments, the thermoresponsive polymer is insoluble in water at a temperature above 32° C., and is soluble in water at a temperature of 32° C. or lower. In some embodiments, the thermoresponsive polymer is insoluble in water at a temperature above 30° C., and is soluble in water at a temperature of 30° C. or lower. In some embodiments, the thermoresponsive polymer is insoluble in water at a temperature above 37° C., and is soluble in water at a temperature of 32° C. or lower.

The therapeutic compound may be any compound providing a therapeutic effect to a subject, including alleviation of symptoms of a disease, reduction of pain or suffering, and inhibition of disease development. In some embodiments, the therapeutic compound comprises one or more anesthetic compounds, or pharmaceutically acceptable salts thereof.

Examples of suitable anesthetic compounds include, but are not limited to, articaine, benzocaine, bupivacaine, chloroprocaine, dibucaine, dimethocaine, etidocaine, lidocaine, mepivacaine, prilocaine, ropivacaine, tetracaine, trimecaine, and pharmaceutically acceptable salts thereof. In particular embodiments, the anesthetic compound is bupivacaine, or a pharmaceutically acceptable salt thereof.

The pharmaceutical composition may include a membrane surrounding the gel. Advantageous, the membrane may prevent rapid diffusion of the thermoresponsive polymer following the gel→sol transition at a low temperature, and the resulting burst release of the therapeutic compounds. In addition, the membrane may maintain a significant “local” concentration of the dissolved thermoresponsive polymer, and may enable a sol→gel transition and re-formation of the gel composition. Advantageously, the membrane may enable repeated transition of the polymer between the gel and the sol states, thus providing repeatable temperature-controlled release of the therapeutic compound from the gel composition.

In some embodiments, the membrane is a nanoporous membrane. For example, the membrane may have an average pore size between 1 nm and 100 nm, such as about 10 nm, about 20 nm, about 30 nm, about 40 nm, about 50 nm, about 60 nm, about 70 nm, about 80 nm, or about 90 nm. In some embodiments, the membrane has an average pore size of about 5 nm to about 95 nm, including for example about 10 nm to about 80 nm, about 20 nm to about 80 nm, about 30 nm to about 80 nm, about 40 nm to about 80 nm, about 10 nm to about 60 nm, about 20 nm to about 60 nm, or about 30 nm to about 60 nm. In some embodiments, the membrane has an average pore size of about 20 nm to about 80 nm, or about 20 nm to about 60 nm. In some embodiments, the membrane has about 10⁷ to about 10⁹ pores/cm², including for example 1×10⁷, 5×10⁷, 1×10⁸, 5×10⁸, or 1×10⁹ pores/cm². In some embodiments, the membrane has about 1×10⁷ pores/cm² to about 1×10⁹ pores/cm², about 5×10⁷ pores/cm² to about 1×10⁹ pores/cm², about 5×10⁷ pores/cm² to about 5×10⁸ pores/cm², about 5×10⁷ pores/cm² to about 1×10⁸ pores/cm², or about 1×10⁸ pores/cm² to about 5×10⁸ pores/cm². In some embodiments, the membrane has about 5×10⁷ pores/cm² to about 5×10⁸ pores/cm². In some embodiments, the membrane includes polycaprolactone (PCL), alginate, or a combination thereof. In some embodiments, the membrane is a nanoporous PCL membranes with a pore sizes of about 20 nm to about 60 nm and approximately 1×10⁸ pores/cm².

In another aspect, the present disclosure also provides an implant device which includes a pharmaceutical composition as described herein. In some embodiments, the implant device include a gel composition having an anesthetic compound dispersed therein and a membrane surrounding the gel composition. The device may have additional structural components to facilitate the administration and maintain the function of the device.

### 3. METHOD

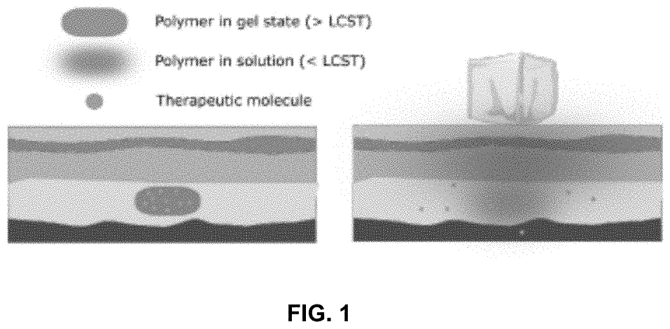

The present disclosure provides a non-invasive method to trigger controlled release of on-demand medication (e.g., pain relief). Several publications describe complex formulations engineered to both sequester therapeutic molecules prior to stimulus (thereby avoiding the toxic effects of excessive administration) and release the molecules at an appropriate rate when triggered. On the other hand, cooling is a highly attractive stimulus for triggering drug release (e.g., for pain management) for several reasons. For example, the application of a cold pack or ice is already a well-established means to achieve some level of short term pain relief both in and out of the clinic, and the tools to achieve cooling (ice, cold pack, etc.) are highly accessible. Further, unlike other external stimuli for drug release, cooling is effectively a removal of energy (removal of heat), as opposed to other external stimuli that are effectively the addition of energy (heat, light, electromagnetic signals, acoustic, etc.) and can thus result in complications and tissue damage if too much energy is delivered. Cooling thus may serve as a simple, low-risk stimulus for inducing drug release, and is practical even in a resource-limited setting (FIG. 1).

Stimuli for the release of therapeutics may come from either an endogenous biological process that results in an environmental change (e.g., change in local reactive oxygen species concentration, pH change, enzymatic activity, change in soluble species concentration) or, alternatively, a stimulus provided by apparatus outside body (e.g., laser irradiation, RF signal, ultrasound, magnetic field) that serves as a remote trigger. A wide range of technologies have been developed to enable on demand, externally triggered release of therapeutics. In particular, a variety of approaches relying upon thermal activation of temperature-responsive systems have been demonstrated; these all depend upon heating induced by an external energy source such as a near-infrared laser or radio frequency signal. While many of these approaches utilize heating to permeabilize thermosensitive liposomes, there are also reported studies describing the use of thermoresponsive polymer gels to release therapeutics upon heating.

Heat-induced reversible deswelling of gels formed from chemically crosslinked thermoresponsive polymers has been used for a wide range of controlled release applications. When a chemically crosslinked thermoresponsive polymer hydrogel is heated above the LCST, it deswells and ejects any absorbed therapeutic molecules along with water molecules.

On the other hand, even without chemical crosslinking, thermoresponsive polymers can form a physically crosslinked gel when heated above the LCST at a high enough concentration (sol->gel transition); this gel disassembles and the polymers may dissolve when cooled below the LCST (gel->sol transition). Instead of using heat-triggered deswelling of chemically crosslinked thermoresponsive polymer gels, the present disclosure uses cooling-triggered disintegration of physically crosslinked thermoresponsive polymer gels (e.g., injectable gels) to deliver a therapeutic compound.

Platforms that enable on-demand, controlled release of anesthetics to enable patient-controlled prolonged duration local anesthesia are advantageous. In particular, there continues to be a significant need for an on-demand anesthetic delivery platform that relies upon a simple, non-invasive, and safe stimulus that patients can use reliably.

In one aspect, the present disclosure provides a method of delivering a therapeutic compound, which includes administering a pharmaceutical composition as described herein to a subject in need thereof.

The pharmaceutical composition as described herein may be administered to a subject in need thereof by a variety of routes known to those skilled in the art, including without limitation oral, intravenous, intramuscular, topical, intradermal, subcutaneous, systemic, and/or intraperitoneal administrations. In some embodiments, the pharmaceutical composition is administered subcutaneously, intradermally, or intramuscularly. In particular embodiments, the pharmaceutical composition is administered subcutaneously (e.g., by injection).

The amount of the therapeutic compound or a pharmaceutically acceptable salt thereof to be delivered (e.g., for treatment of a condition or disease) will vary not only with the particular compound or salt selected but also with the route of administration, the nature and/or symptoms of disease or condition being treated and the age and condition of the patient, and will be ultimately at the discretion of the attendant physician or clinician.

A suitable dose for the therapeutic compound or a pharmaceutically acceptable salt thereof to be delivered may be in the range of from about 0.01 mg/kg to about 100 mg/kg, such as from about 0.05 mg/kg to about 10 mg/kg. For example, a suitable dose may be in the range from about 0.10 mg/kg to about 7.5 mg/kg of body weight per day, such as about 0.10 mg/kg to about 0.50 mg/kg of body weight of the recipient per day, about 0.10 mg/kg to about 1.0 mg/kg of body weight of the recipient per day, about 0.15 mg/kg to about 5.0 mg/kg of body weight of the recipient per day, about 0.2 mg/kg to 4.0 mg/kg of body weight of the recipient per day. The compound may be administered in unit dosage form; for example, containing 1 to 100 mg, 10 to 100 mg or 5 to 50 mg of active ingredient per unit dosage form.

The determination of effective dosage levels to achieve the desired result, may be accomplished by one skilled in the art using routine methods, for example, human clinical trials, in vivo studies and in vitro studies. For example, useful dosages of a compound or pharmaceutically acceptable salts thereof may be determined by comparing their in vitro activity and in vivo activity in animal models. Such comparison may be done by comparison against an established drug.

The method may further include reducing the temperature of the skin of the subject, thereby causing the thermoresponsive polymer to become soluble in water and releasing the therapeutic compound.

In some embodiments, the therapeutic compound administered by the present method is an anesthetic compound, or a pharmaceutically acceptable salt thereof as described herein. In particular embodiments, the therapeutic compound is bupivacaine, or a pharmaceutically acceptable salt thereof.

In some embodiments, the composition administered by the present method includes a membrane surrounding the gel. For examples, the member may be a nanoporous membrane including polycaprolactone, alginate, or a combination thereof as described herein. In particular embodiments, the present method includes administering a gel composition as described herein, comprising a therapeutic compound comprising bupivacaine or a pharmaceutically acceptable salt thereof, and a membrane surrounding the gel.

In some embodiments, after the initial cooling and release of the therapeutic compound, the present method further includes increasing the temperature (e.g., temperature of the skin), thereby causing the thermoresponsive polymer of the composition to become insoluble in water. As a result, the thermoresponsive polymer may again transition to gel state, thereby re-encapsulating the therapeutic compound in the re-formed hydrogel and effectively stopping the release of the therapeutic compound. For example, the rate of the release at the increased temperature may be less than 10%, less than 7.5%, less than 5%, less than 4%, less than 3%, less than 2%, less than 1%, less than 0.5%, or even less than 0.1% of the rate of the initial release. The pharmaceutical composition as described herein may advantageously enable repeated transition of the thermoresponsive polymer between the gel and the sol states, thus providing repeatable temperature-controlled release of the therapeutic compound. Accordingly, in some embodiments, the present method further include repeating the process of reducing and increasing the temperature (e.g., temperature of the skin), thereby providing repeated release of the therapeutic compound from the gel composition.

Advantages

In some embodiments, the disclosed methods may be carried out by simple application of ice to the skin. For example, the application of ice may induce significant temperature decrease at a depth of 1.5-2 cm beneath the skin surface, reaching temperatures below 30° C. in under 30 minutes. Thus, a decreased temperatures achieved within tissue by ice application may be below the LCST of many thermoresponsive polymers, such as PNIPAM, and polyvinyl caprolactam and its copolymers (with LCST tunable in the range of about 32° C. to about 40° C.). To achieve the desired temperature-dependent response, the LCST of a thermoresponsive polymer may be tuned by copolymerization with appropriate comonomers. Alternatively, simply adjusting the depth below the skin at which the implant device or composition disclosed herein is injected may also achieve the desired response (as tissue closer to the ice at the skin surface are colder than tissue at greater depth from the skin surface).

Moreover, the present method may be advantageously employed for pain relief and anesthetics delivery applications. Previously reported cold-responsive nanoparticle or nanogel formulations only demonstrate irreversible gel->sol transition, likely because of the small size of the nanoparticles and low local concentration. These formulations resulted in a rapid decrease in local polymer concentration upon disintegration due to surface-to-volume ratio of the nanoparticles, and thus only a single burst release of therapeutics was possible. Remarkable, the compositions described herein may be capable of reversible gel->sol transition, because the local concentration may be largely maintained (i.e. polymer located in a confined environment such as a vial). Thus, the present compositions and delivery vehicles may be particularly beneficial for pain relief applications, where it is highly desirable to enable multiple releases of the medication (e.g., triggered by cooling over a period of time) from a single injection. Thus, the methods described herein may enable cooling-triggered release a therapeutic from a physically crosslinked, thermogelling injectable polymer delivery vehicle. In particular, the use of cooling to trigger release of an anesthetic for pain management is advantageous due to the familiarity of using cold to temporarily numb tissue, and because of the reduced complexity of using cooling as compared to other stimuli.

In another aspect, the present disclosure provides a method of inducing a chemical reaction, the method comprising
reducing a temperature of a reaction system comprising:
   a first gel comprising a thermoresponsive polymer and a first reactant in the first gel; and
   a second gel comprising a thermoresponsive polymer and a second reactant in the second gel;
   wherein each polymer is soluble in water below a lower critical solution temperature (LC ST);
thereby causing each gel to transition to a sol state and mix, thereby allowing the first reactant and the second reactant to mix, and thereby initiating the chemical reaction.

In some embodiments, a first and a second thermoresponsive polymers described form herein separately form two physically crosslinked thermoresponsive gels, each containing a separate reactant species. In some embodiments, the gels may be placed next to each other. For example, the two gels may be separated by a thin barrier gel that contains no additional species. At elevated temperature, the entire system remains in the gel state, and the reactant species are sequestered inside the gels and do not interact. Upon cooling below the LCST, the gels undergo disintegration and form solutions, at which point the reactants will interact and the reaction will take place. In some embodiments, the gelling process is reversible, but the separate sequestration of the reactants is not reversible, and the reaction cannot be reversed upon subsequent heating.

In some embodiments, the reaction system described herein may be used as indicators that an environment has cooled below a threshold temperature, or as autonomous systems to produce a product (e.g., heat from an exothermic reaction, deliver drugs) upon cooling. For example, a colorimetric change or other indicators such as gas bubbles from mixing a carbonate reactant (e.g., baking soda) and an acidic reactant (e.g., vinegar), may be employed to show the initiation and progress of the reaction upon cooling.

### 4. EXAMPLES

### Example 1. In Vitro Stability and Cooling-Triggered Release Profile of a Model Drug Sequestered in Physically Crosslinked Thermoresponsive Gel

Thermoresponsive Behavior of Polymers

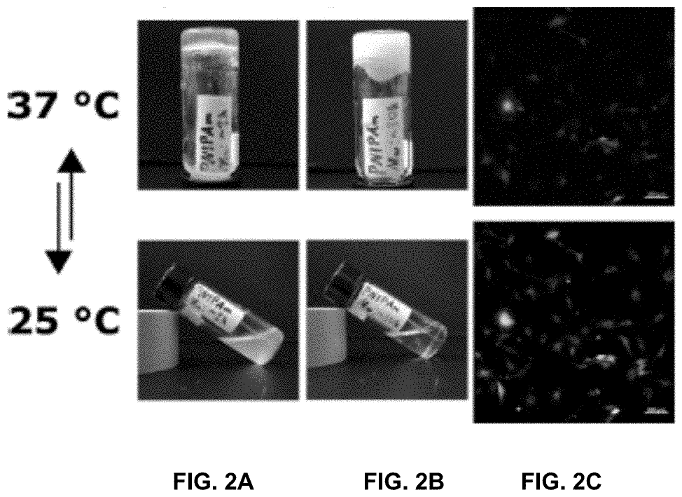

Commercially available thermoresponsive polymers, such as PNIPAM of various molecular weights and including various comonomers from Sigma Aldrich, polyvinyl caprolactam from BASF, are studied for their thermoresponsive behavior suitable as a cooling-triggered delivery vehicle. Typical results of PNIPAM demonstrates physically crosslinked gel state at a temperature above LCST (FIG. 2A) and a solution state at a temperature below LCST (FIG. 2B). Initial in vitro experiments indicates a temperature-dependent delivery of a hydrophobic compound (Nile Red) a to cells in culture (FIG. 2C) by diffusion from the gel (pre-trigger) to sol (post trigger) polymer phases. These results verifies that a thermoresponsive polymer can be induced to disintegrate in tissue when the tissue has been cooled below the LCST of the polymer system.

Cooling-Triggered Delivery of Nile Red

An in vitro experiments is conducted to characterize temperature-dependent release of a model small molecule payload, Nile Red. Nile Red (Mw 318 g/mol), which has a similar molecular weight to an anesthetic, bupivacaine (Mw 288 g/mol), and both compounds are highly lipophilic. Hydrogels of poly(N-isopropylacrylamide) (PNIPAM) (40 kDa, 5% w/v) containing Nile Red (800 ng/mL or 2.4 μg/mL) are prepared. Specifically, PNIPAM (Sigma-Aldrich) is dissolved in PBS at room temperature (below the LCST). Nile Red is then added, and the formulation is sterile filtered, cast into molds, and subsequently warmed in a 37° C. humidified incubator (above the LCST) to form a macroscopic, physically-crosslinked, dye-loaded hydrogel.

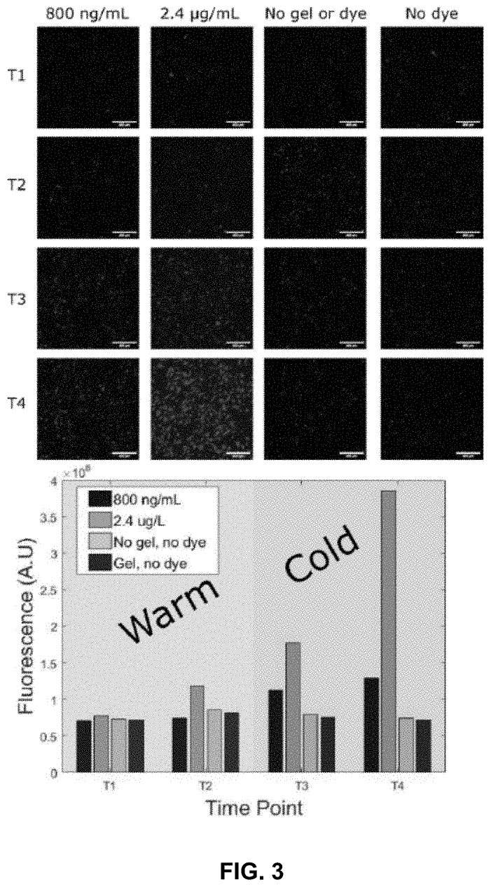

The gel samples are introduced to human umbilical vein endothelial cells cultured in 6-well plates in a 37° C. humidified incubator with 5% CO₂. The lipophilic fluorophore Nile red intercalates into the cell membrane upon release. Fluorescence imaging is measured at four sequential time points: T1—immediately after adding warm gel to cell culture, T2—after 24 hours of culture in 37° C., T3—after removing from incubator and equilibrating at room temperature for 30 min, and T4—after removing from incubator and equilibrating at room temperature for 60 min. As shown in FIG. 3, stable dye retention is demonstrated at the 24-hour time point (T2), and release occurs upon cooling (T3, T4). These results show the in vitro release of Nile Red upon cooling from 37° C. to room temperature, demonstrating that a payload can, in fact, be stably bound in a physically crosslinked gel and released upon cooling-triggered disintegration

PNIPAM of different average molecular weights (e.g., Mw 10 kDa, 40 kDa, or 85 kDa) and different LCST are tested according to the above process to demonstrate various properties of the gel and solution phases (above and below the LCST) for each polymer, including, for example, mechanical and diffusion properties in the gel and solution states and the amount of payload that can be sequestered in the gel.

In some experiments, each gel are prepared at 300 μL total volume, and has two different geometries: disc (5 mm radius, 4 mm height) and rod (1 mm radius, 10 mm length). The gels are investigated to determine the ease and reproducibility of fabrication and encapsulation in a nanoporous membrane. Holding the total volume constant, the concentration of both the polymer (0.1%-10% w/v) and the Nile Red (0.1%-5% relative to polymer mass) are varied to determine the loading efficiency and maximum loading. Additionally, the dye-loaded polymer gels are washed 3× in PBS and incubated overnight in PBS at 37° C. The amount of dye remaining in the gel is quantified using a plate reader to determine whether loading level has an effect on non-triggered dye leakage form the gel. Rheological properties of each formulation are characterized using an AR-G2 rheometer. The diffusion coefficient of Nile Red in the gel (OFF) and solution (ON) states are quantified for all formulations using fluorescence recovery after photobleaching (FRAP) to maximize the ON/OFF ratio.

In some experiments, blends of PNIPAM and other materials (e.g. dextran or PEG), or copolymers of N-isopropylacrylamide (NIPAM) and other monomers such as acrylic acid or PEG, or Pluronics are tested as thermoresponsive polymers suitable for cooling-triggered release of therapeutics. In some experiments, a “plum-pudding” approach may be employed to reduce leakage rate, in which a two-phase material is used and the thermoresponsive gel is used to temporarily sequester drug-laden micro- or nanoparticles.

Formulations with Nanoporous Membrane

To avoid potential systemic toxicity, a nanoporous membrane surrounding the thermoresponsive gel or implant is tested to demonstrate the lack of significant burst release and control over payload release rate. The gels are surrounded by a rate-controlling membrane that restricts transport to prevent burst release of the payload as well as avoid dilution of the thermoresponsive polymer when in the liquid state (which would inhibit re-gelling and thus repeated dosing). This membrane will also inhibit host tissue integration with and infiltration into the thermoresponsive implant.

In some experiments, thin nanoporous polycaprolactone (PCL) films are employed. PCL membranes have the advantage that they are bioresorbable and thus no surgical removal would be needed when the depot is depleted. The film thickness is quantified by profilometry, and the pore size (e.g., about 20-60 nm) and density (e.g., about 10⁸ pores/cm²) are quantified using scanning electron microscopy or atomic force microscopy. The modulation of the structural parameters of the membrane (e.g., varying membrane porosity) achieves a range of overall device release rates. The trans-membrane flux of Nile Red dissolved in PBS and of Nile Red in room temperature PNIPAM solution are quantified using nanoporous membranes attached to the bottom of Transwell inserts. The resulting data are modeled and exhibit first-order behavior as the diffusing molecule is substantially smaller than the pore size. To produce complete devices, sterile membranes are formed in a biological safety cabinet, cut to size, wrapped around gels, and heat sealed.

In some experiments, formulations with alginate membranes are prepared, for example, by formulating the PNIPAM thermoresponsive polymer in a solution containing calcium and subsequently placing the gels (above the LCST) in an alginate solution. The calcium leaks out and causes crosslinking of the alginate around the PNIPAM. This approach may enable the use of thermoresponsive microgels (and thus higher surface area for release), as manual coating/sealing of the membrane would not be needed. Other possible nanoporous membranes for release rate control include those formed from known block copolymers (Jackson et al., Nanoporous Membranes Derived from Block Copolymers: From Drug Delivery to Water Filtration. ACS Nano, 2010, 4, 3548-3553, incorporated herein by reference in its entirety).

Device Characterization

Physically crosslinked thermoresponsive gels loaded with dye, and dye-laden gels encapsulated by a nanoporous rate-controlling membranes are incubated in a slowly stirred PBS bath at 37° C. and additionally in the vicinity of GFP-expressing fibroblasts in culture to visualize local release of the model drug. For experiments in PBS, the concentration of dye in solution are measured every hour for 10 days (to characterize stability and determine OFF release rate), after which the solution are allowed to cool to room temperature while measuring temperature and dye concentration in solution every 15 minutes (to determine ON release rate). Device release kinetics are analyzed using the Weibull empirical model to capture behavior of more than the first 60% of the release curve and provide insight into the dominant release mechanism (e.g., Fickian diffusion-controlled) of this reservoir/membrane system. For each formulation, the ON/OFF release rate ratio are used as a figure of merit (this may be concentration dependent if the release is not zero order). For cell culture experiments, cells near the dye-laden device are imaged with a confocal microscope (Zeiss LSM 710) every day for 14 days while maintained at 37° C., and subsequently every 15 minutes while the temperature of a microscope-mounted incubation chamber is slowly reduced from 37° C. to room temperature (temperature will be recorded at each imaging timestamp).

In some experiments, without the membrane surrounding the thermoresponsive delivery vehicle, cooling induces a burst release of the sequestered dye, while prior to cooling some small amount of leakage into the surrounding solution is observed. In this configuration, repeated gelling of the thermoresponsive polymer is almost impossible. When the dye-laden polymer is encapsulated in the nanoporous membrane, however, the local concentration of polymer remains roughly constant, minimal leakage of dye is observed when incubated at elevated temperatures, and a slow release is observed when the solution is cooled below the LCST. In some experiments, an ON/OFF ratio of at least 50, an OFF leakage rate less than 1% initial payload mass per day, and/or an ON release rate of −0.5 mg/hr is achieved.

### Example 2. In Vivo Stability and Release of Bupivacaine from a Physically Crosslinked Thermoresponsive Gel to Achieve Cooling-Triggered Prolonged Duration Local Anesthesia

Sterile devices are produced as described in Example 1, loaded with either Nile Red (model drug, to characterize in vivo release profiles) or bupivacaine (15 mg/mL). Three highest ON/OFF ratio formulations with OFF leakage rates below 1% initial payload mass per day are employed for in vivo Nile Red studies. The formulation that performs best during in vivo Nile Red studies (as determined by ON/OFF ratio and leakage rate) are used for in vivo studies with bupivacaine.

Nile Red- or bupivacaine-loaded gels are implanted in adult Sprague-Dawley rats alongside a wireless temperature probe. Stability are characterized using dye-loaded formulations, with animals imaged daily to determine the extent to which the dye leaks from the gelled implant; quantitative data are derived from depletion of the Nile Red fluorescence signal. To characterize release, rats implanted with dye-loaded formulations are subjected to a cooling stimulus (ice applied to the skin while tissue temperature is remotely monitored), and subsequently imaged to determine the extent to which cooling triggers release of the sequestered compound in vivo as observed by decrease in the depot fluorescence intensity. Multiple cycles of cooling are performed (with a 24-hour recovery period between cycles) until no significant change in the fluorescence signal from the depot is observed.

To demonstrate cooling-triggered release of an anesthetic, animals implanted with bupivacaine-loaded devices undergo neurobehavioral testing as previously described (Rwei et al., Ultrasound-triggered local anaesthesia, Nat. Biomed. Eng. 2017, 1, 644-653) both before and after a cooling stimulus. Multiple cooling cycles are used to determine the extent to which the cooling-triggered bupivacaine release is repeatable; for these studies a 24-hour recovery period between the end of a nerve block and the next cooling trigger are used. Each animal is implanted with a drug-free device in the right leg as a control; this serves to separate out the numbing effects of cooling itself from the drug-induced local anesthesia. An additional experimental group is injected with 15 mg/mL bupivacaine in sterile saline (instead of implanted with a device) to establish a baseline nerve block duration (approximately ˜200 min). The difference between the duration of nerve blocks due to injection of bupivacaine solution and the duration of blocks caused by cooling-triggered release from an implanted device is determined. To investigate tissue response to the implanted devices, after the animals are sacrificed the tissue surrounding the sciatic nerve is removed, processed for histology (H&E staining), and analyzed via optical microscopy.

Prior to any cooling, the loaded dye or drug remains within the gel with minimal leakage to nearby tissue (leakage rates as low as, or lower than, those measured for identical formulations in vitro). Local cooling of the implantation site causes the gel to liquify and enables efficacious release of the dye or drug through the nanoporous membrane; subsequent removal of the cooling stimulus and equilibration of the tissue to physiological temperature reduce the release rate back to the leakage rate. This process is repeatable until the reservoir is depleted of the payload. In some studies, at least 10 cycles of cooling triggered anesthetic release, each causing at least 3 hours of nerve block, are achieved.

Depending on the composition of the rate-limiting membrane, there may be an immune response leading to fibrotic capsule formation, which may be reduced using known methods for both PCL and alginate. In addition, copolymers of NIPAM and acrylic acid are used, as the presence of more —COO— promotes stronger interaction with cationic compounds such as bupivacaine.

It is understood that the foregoing detailed description and accompanying examples are merely illustrative and are not to be taken as limitations upon the scope of the invention, which is defined solely by the following claims.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A pharmaceutical composition comprising:
a gel comprising a thermoresponsive polymer, the polymer being soluble in water at a temperature of 37° C. or lower; and
a therapeutic compound, or a pharmaceutically acceptable salt thereof, dispersed in the gel.

2. The pharmaceutical composition of claim 1, wherein the polymer comprises a monomer selected from the group consisting of N-alkyl acrylamide, N,N-dialkyl acrylamide, N-acryloylpyrrolidine, N-acryloylpiperidine, N-vinyl caprolactam, N-vinyl propylacetamide, N-vinyl-5-methyl-2-oxazolidone, N-vinyl isobutyramide, N-acryloyl-L-proline methyl ester, N-acryloyl-4-trans-hydroxy-L-proline methyl ester, methyl 2-propionamidoacrylate, methyl 2-isobutyracrylate, acrylic acid, glucose, O-methyl glucose, lactic acid, glycolic acid, vinyl acetate, ethylene glycol, propyleneoxide, vinylether, alkylglycidylethers, phosphoester, and a combination thereof.

3. The pharmaceutical composition of claim 2, wherein the polymer comprises a monomer selected from the group consisting of N-ethylacrylamide, N-ethylmethacrylamide, N,N′-ethylmethylacrylamide, N,N′-diethylacrylamide, N-n-propylacrylamide, N-n-propylmethacrylamide, N-isopropylacrylamide, N-isopropylmethacrylamide, and a combination thereof.

4. The pharmaceutical composition of claim 1, wherein the polymer is a homopolymer.

5. The pharmaceutical composition of claim 1, wherein the polymer is a copolymer.

6. The pharmaceutical composition of claim 2, wherein the polymer comprises poly(N-isopropylacrylamide).

7. The pharmaceutical composition of claim 1, wherein the polymer comprises poly(N-isopropylacrylamide) having an average molecular weight (Mw) of about 1 kDa to about 100 kDa.

8. The pharmaceutical composition of claim 1, wherein polymer is soluble in water at a temperature of 32° C. or lower.

9. The pharmaceutical composition of claim 1, wherein polymer is insoluble in water above 32° C.

10. The pharmaceutical composition of claim 1, wherein the therapeutic compound comprises an anesthetic compound or a pharmaceutically acceptable salt thereof.

11. The pharmaceutical composition of claim 10, wherein the anesthetic compound comprises articaine, benzocaine, bupivacaine, chloroprocaine, dibucaine, dimethocaine, etidocaine, lidocaine, mepivacaine, prilocaine, ropivacaine, tetracaine, trimecaine, a pharmaceutically acceptable salt thereof, or a combination thereof.

12. The pharmaceutical composition of claim 11, wherein the anesthetic compound comprises bupivacaine, or a pharmaceutically acceptable salt thereof.

13. The pharmaceutical composition of claim 1, further comprising a membrane surrounding the gel.

14. The pharmaceutical composition of claim 13, wherein the membrane is a nanoporous membrane.

15. The pharmaceutical composition of claim 13, wherein the membrane comprises polycaprolactone, alginate, or a combination thereof.

16. An implant device comprising a pharmaceutical composition of claim 1.

17. A method of delivering a therapeutic compound, the method comprising:
administering the pharmaceutical composition of claim 1 to a subject in need thereof.

18. The method of claim 17, wherein the pharmaceutical composition is administered subcutaneously, intradermally, or intramuscularly.

19. The method of claim 17, wherein the subject has skin and the temperature of the skin is reduced, thereby causing the thermoresponsive polymer to become soluble in water and releasing the therapeutic compound.

20. The method of claim 19, further comprising
increasing the temperature of the skin, thereby causing the polymer to become insoluble in water; and
optionally repeating the process of reducing and increasing the temperature of the skin.

21. The method of claim 17, wherein the therapeutic compound is an anesthetic compound, or a pharmaceutically acceptable salt thereof.

22. The method of claim 17, wherein the pharmaceutical composition further comprises a membrane surrounding the gel.

23. A method of inducing a chemical reaction, the method comprising
reducing a temperature of a reaction system comprising:
a first gel comprising a thermoresponsive polymer and a first reactant in the first gel; and
a second gel comprising a thermoresponsive polymer and a second reactant in the second gel;
wherein each polymer is soluble in water below a lower critical solution temperature (LCST);
thereby causing each gel to transition to a sol state and mix, thereby allowing the first reactant and the second reactant to mix, and thereby initiating the chemical reaction.
